(19) United States
(12) Patent Application Publication
(10) Pub. No.: US 2003/0044311 A1
(43) Pub. Date: Mar. 6, 2003

(54) Applications for use of pulsed light

(76) Inventors: John Sousa, Hales Location, NH (US); Louis R. Panico, Danvers, MA (US); C. Richard Panico, Medford, MA (US)

Correspondence Address: HALE AND DORR, LLP, 60 STATE STREET, BOSTON, MA, 02109

(21) Appl. No.: 10/190,349

(22) Filed: Jul. 3, 2002

Related U.S. Application Data
(60) Provisional application No. 60/303,308, filed on Jul. 6, 2001.

(51) Int. Cl. A61L 2/08; B65B 55/08; C01B 21/02

(52) U.S. Cl. 422/22; 422/23; 426/234; 204/157.46

(57) ABSTRACT

Pulsed ultraviolet light is used to address contamination by various microorganisms in different materials provided to people or animals, including food, water, and blood by transfusion.

### CROSS REFERENCE TO RELATED APPLICATIONS

This application claims priority from provisional application No. 60/303,308, filed Jul. 6, 2001, which is expressly incorporated by reference.

### BACKGROUND

Research in various areas is ongoing to address the problem of contamination by various microorganisms in different materials provided used by people or animals, including food, water, and blood by transfusion. There is a need for a simple, yet effective method of disinfection or deactivation of objectionable pathogens in a reliable manner.

### SUMMARY

The present invention relates to disinfection and/or deactivation of microorganisms. This is accomplished through the use of pulsed ultraviolet (UV) light as a sterilization or disinfection technique, preferably with the operating parameters as cited below, using a commercially-available pulse light system with a mercury-free xenon lamp. Other features will be apparent from the following detailed description, drawings, and claims.

### DETAILED DESCRIPTION

In one aspect, the present invention includes a method for eliminating the likelihood that the blood plasma is a resident host to microorganisms. This is accomplished through the use of pulsed ultraviolet (UV) light as a sterilization technique for blood banks. The assumption is made that adequate pre-treatment procedures are in place and operating in tandem with this UV technology.

The light output of pulsed xenon lamps in a pulsed light apparatus renders pathogenic (disease causing) microorganisms incapable of reproducing with rapidity and effectiveness. When biological organisms are exposed to pulsed UV light, it is the generic material or deoxyribonucleic acid (DNA) that is affected by the penetration of light through cell walls and causes molecular rearrangement of the microorganism's DNA, thus preventing reproduction (cell mitosis) by cross-linking the DNA. Due to different cell makeup, different levels of UV energy are required for destruction. These levels can be established through experimentation.

The specific blood components used for transfusions, platelets, plasma, and red blood cells do not contain DNA or RNA, and therefore retain their biological activity after exposure.

To date, known microorganisms are generally destroyed with proper UV exposure. Xenon Corporation's XL mercury-free pulsed light system provides a sufficient amount of UV radiation, resulting in an effective anti-microbiological process, which illustrates the point that inclusion of a pulsed UV system in the treatment of blood offers an effective sterilization means.

In another aspect, the invention includes a method for using pulsed light on fruit or other food item for surface sterilization and/or for treating a protective shrink-wrap on the food item.

A pulsed light apparatus inhibits fungal growth on fruits or vegetables by exposure to an effective amount of pulsed UV light, so as to inhibit the production of mycotoxins on plants and crops without compromising the post harvest quality (firmness, texture, aroma and color) and the use of pulsed light to encase the individual fruits or vegetables in a shrink-wrap film by shrinking the film with pulsed aseptic light, preventing further attack by harmful microorganisms. The treatment of the shrink wrap can be in addition to treatment of the fruit or other food item itself with pulsed light.

In another aspect, current methods for water sterilization can be improved upon by the use of pulsed light tuned to the specific need of the water for deep sterilization and or rapid sterilization.

A mercury-free pulsed light apparatus provides instantaneous, high intensity pulsed ultraviolet light capable of delivering the required amount of quantum energy sufficient to break chemical bonds and is capable of killing most microorganisms that it comes in contact with. All significant water pathogenic microorganisms are destroyed, thus disinfecting the water supply.

Additionally, the pulsed UV radiation breaks down toxic ammonia from fish wastes (which contaminates the water hatchery ecosystem), into nitrate that is similar to the results of the reaction of ammonia→nitrite→nitrate in the final stage of the natural biological metabolic waste conversion, known as the “Nitrogen Cycle.” Nitrate, the final product, is what plants utilize as fertilizer, thus removing them from water.

The methods according to the embodiments of the present invention can be implemented using know pulse UV technology. Typical systems include a power supply, energy storage (such as with capacitors), a pulse configurator, and a lamp assembly with related optics. These are all known components of pulsed light systems, including the XL system referenced above.

Exemplary operating parameters are as follows:

Pulse Duration: 1-1,000 microseconds measured at ⅓ peak value.

Energy per Pulse: 1-2,000 joules.

Pulse Recurrence Frequency: Zero (0) to ten (10) pulses per second.

Exposure Time: 0.1 to 100 seconds, or single pulse to continuous pulsing.

Lamp Configuration: (shape): linear or spiral.

Spectral Output: 100-1,000 nanometers.

Lamp Cooling: ambient, forced air, or water.

Wavelength Selection: (external to the lamp): none or IR filter.

Lamp Housing Window: quartz, suprasil, or sapphire.

Sequencing: Burst mode, synchronized burst mode, or continuous running.

The pulse duration noted above can be reduced to below 1 microsecond if desired, and is preferably on the order of 1-10 microseconds.

Having described embodiments of the invention, it should be apparent that modifications can be made without departing from the scope of the invention as defined by the appended claims.

What is claimed is:

1. A method comprising providing an effective amount of light from a mercury-free xenon pulsed lamp to disinfect and/or decontaminate microorganisms in one of water, food, or blood, the method further including providing light with pulses on the order of 1-10 microseconds.

2. A method comprising providing an effective amount of light from a mercury-free xenon pulsed lamp to a shrink wrapping around a food item to disinfect and/or decontaminate microorganisms on the wrapping.

3. The method of claim 2, further comprising providing an effective amount of light from a mercury-free xenon pulsed lamp to the food item before the shrink wrapping to disinfect and/or decontaminate microorganisms on the food item.

4. A method comprising providing an effective amount of light from a mercury-free xenon pulsed lamp to break down toxic ammonia from fish wastes into nitrate.
